(19) United States
(12) Patent Application Publication
(10) Pub. No.: US 2007/0044254 A1
(43) Pub. Date: Mar. 1, 2007

(54) Novel 2,6-disubstituted primary para-phenylenediamines and process for their use in the oxidation dyeing of keratin fibers

(76) Inventors: Laure Ramos-Stanbury, Sceaux (FR); Stephane Sabelle, Paris (FR); Thy-My Ly-Carry, Saint Maur des Fosses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/507,445

(22) Filed: Aug. 22, 2006

Related U.S. Application Data
(60) Provisional application No. 60/780,056, filed on Mar. 8, 2006.

(30) Foreign Application Priority Data
Aug. 22, 2005 (FR) 0508666

(51) Int. Cl. A61K 8/42; A61K 8/49; C07D 207/04; C07D 295/04

(52) U.S. Cl. 8/406; 548/577; 564/156; 564/373; 564/443; 562/433; 548/335.1

(57) ABSTRACT

Disclosed herein are 2,6-disubstituted para-phenylenediamine compounds chosen from compounds of formula (I) and the salts and solvates thereof: 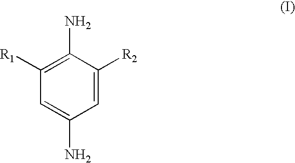 Also disclosed herein are the corresponding intermediate compounds. Further disclosed herein is a composition for the oxidation dyeing of keratin fibers, for example, human keratin fibers such as the hair, comprising at least one para-phenylenediamine compound. Still further disclosed herein are oxidation dyeing processes comprising applying the composition to keratin fibers.

This application claims benefit of U.S. Provisional Application No. 60/780,056, filed Mar. 8, 2006, the contents of which are incorporated herein by reference. This application also claims benefit of priority under 35 U.S.C. § 119 to French Patent Application No. FR 05 08666, filed Aug. 22, 2005, the contents of which are also incorporated herein by reference.

Disclosed herein are novel 2,6-disubstituted primary para-phenylenediamines, compositions for the oxidation dyeing of keratin fibers, for example, human keratin fibers such as the hair, comprising at least one 2,6-disubstituted primary para-phenylenediamine as oxidation base. Also disclosed herein is a process for oxidation dyeing of keratin fibers comprising applying a composition of the present disclosure to the keratin fibers.

It is known practice to dye keratin fibers, for example, human hair with dye compositions comprising at least one oxidation dye precursor, for instance, ortho- or para-phenylenediamines, ortho- or para-aminophenols, and heterocyclic compounds such as diaminopyrazole derivatives, which are generally known as oxidation bases. Oxidation dye precursors, or oxidation bases, are colorless or weakly colored compounds which, when combined with oxidizing products, can give rise to colored compounds and dyes via a process of oxidative condensation.

It is also known that the shades obtained with these oxidation bases can be varied by combining them with couplers or coloration modifiers, the latter being chosen, for instance, from aromatic meta-diamines, meta-aminophenols, meta-diphenols, and heterocyclic compounds.

The variety of molcules available as oxidation bases and couplers allows a wide range of colors to be obtained.

The “permanent” coloration obtained by means of these oxidation dyes ideally satisfies at least one of a number of conditions. For example, the coloration ideally has no toxicological drawbacks, allows shades to be obtained in the desired intensity, shows good fastness with respect to external agents (for example, light, bad weather, washing, permanent waving, perspiration, and/or rubbing), allows grey hair to be covered, and/or is as unselective as possible, i.e., produces the smallest possible coloration differences along the same keratin fiber, which may be differently sensitized (i.e., damaged) between its end and its root. The coloration ideally shows good chemical stability in the formulations, has a good toxicological profile, and/or gives chromatic shades on the hair.

The present inventors have discovered that it is possible to obtain dyes capable of giving strong, sparingly selective colorations that may show excellent properties of resistance to at least one of the various attacking factors to which keratin fibers may be subjected, by using as oxidation base at least one 2,6-disubstituted primary para-phenylenediamine chosen from compounds of formula (I) and the physiologically acceptable salts and solvates thereof.

Disclosed herein are thus novel 2,6-disubstituted primary para-phenylenediamines of formula (I):

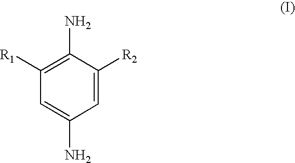

wherein:

R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;

with the proviso that the compounds of formula (I) are not chosen from:

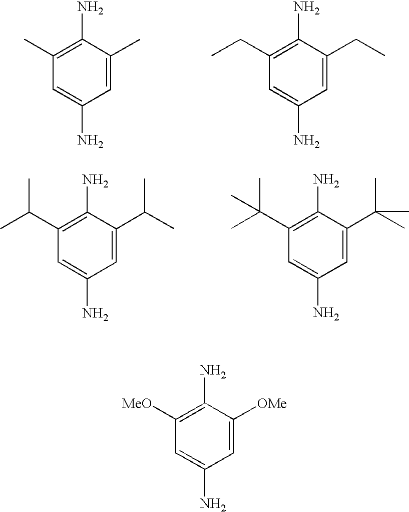

Also disclosed herein are the physiologically acceptable solvates and organic or mineral acid salts of the compounds of formula (I).

The addition salts that may be used in accordance with the present disclosure may be chosen from acid addition salts, such as hydrochloric acid, hydrobromic acid, sulfuric acid, citric acid, succinic acid, tartaric acid, lactic acid, methanesulfonic acid, para-toluenesulfonic acid, benzenesulfonic acid, phosphoric acid, and acetic acid.

The compounds of formula (I) may also be in the form of solvates, for example, hydrates and solvates of linear or branched alcohols such as ethanol and isopropanol.

According to one embodiment, R₁ and R₂, which may be identical or different, are chosen from:

methyl, ethyl, n-propyl, isopropyl, sec-butyl, isobutyl, tert-butyl, (2,2′dimethyl)butyl, imidazopropyl, and dimethylaminopropyl radicals;

methoxybutyl, aminomethyl, (3-dimethylamino)propyl, imidazolo-n-propyl, and pyrrolidinoethyl radicals;

methoxy, hydroxymethyl, α-hydroxyethyl, p-hydroxyethyl, and β-hydroxyisopropyl radicals;

carboxylic radicals; and

amido radicals.

Para-phenylenediamines of formula (I) may include, but are not limited to, the following compounds and the salts and/or solvates thereof:

| | |
|---|---|
| 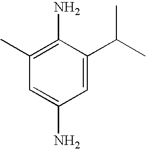 | 2-Isopropyl-6-methylbenzene-1,4-diamine |
| 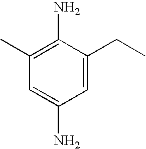 | 2-Ethyl-6-methylbenzene-1,4-diamine |
| 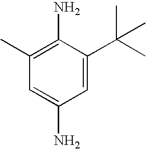 | 2-tert-Butyl-6-methylbenzene-1,4-diamine |
| 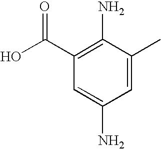 | 2,5-Diamino-3-methylbenzoic acid |
| 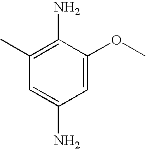 | 2-Methoxy-6-methylbenzene-1,4-diamine |
| 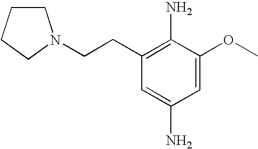 | 2-Methoxy-6-(2-pyrrolidin-1-ylethyl)-benzene-1,4- diamine |
| 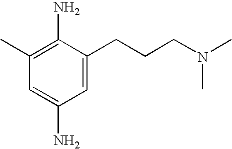 | 2-(3-Dimethylaminopropyl)-6-methyl-benzene- 1,4-diamine |
| 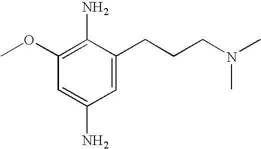 | 2-(3-Dimethylaminopropyl)-6-methoxybenzene- 1,4-diamine |
| 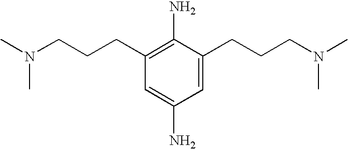 | 2,6-Bis-(3-dimethylaminopropyl)benzene-1,4- diamine |
| 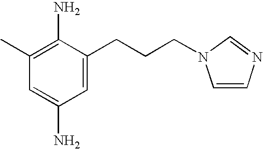 | 2-(3-Imidazol-1-ylpropyl)-6-methyl- benzene-1,4-diamine |
| 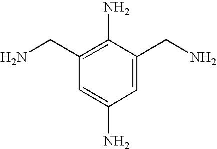 | 2,6-Bisaminomethylbenzene-1,4-diamine |
| 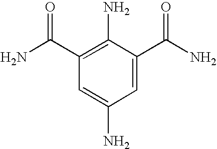 | 2,5-Diaminoisophthalamide |
| 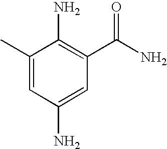 | 2,5-Diamino-3-methylbenzamide |
| 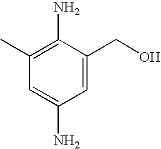 | (2,5-Diamino-3-methylphenyl)methanol |
| 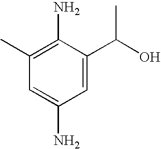 | 1-(2,5-Diamino-3-methylphenyl)ethanol |
| 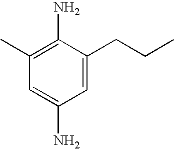 | 2-Methyl-6-propylbenzene-1,4-diamine |
| 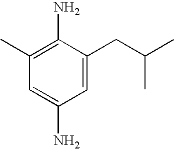 | 2-Isobutyl-6-methylbenzene-1,4-diamine |
| 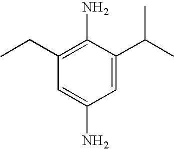 | 2-Ethyl-6-isopropylbenzene-1,4-diamine |
| 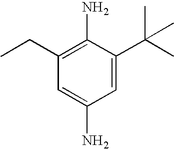 | 2-tert-Butyl-6-ethylbenzene-1,4-diamine |
| 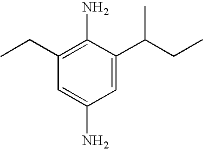 | 2-sec-Butyl-6-ethylbenzene-1,4-diamine |
| 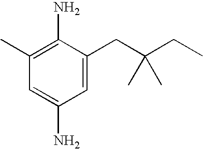 | 2-(2,2-Dimethylbutyl)-6-methyl- benzene-1,4-diamine |
| 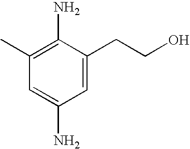 | 2-(2,5-Diamino-3-methylphenyl)ethanol |
| 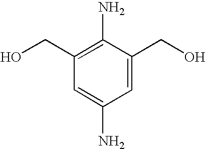 | (2,5-Diamino-3-hydroxymethyl-phenyl)methanol |
| 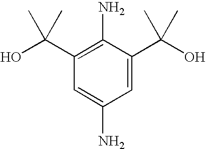 | 2-[2,5-Diamino-3-(1-hydroxy-1-methyl- ethyl)phenyl]propan-2-ol |
| 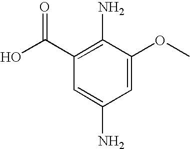 | 2,5-Diamino-3-methoxybenzoic acid |
| 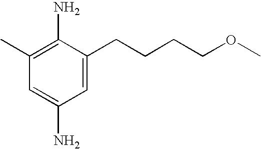 | 2-(4-Methoxybutyl)-6-methylbenzene-1,4-diamine |

In at least one embodiment, the at least one 2,6-substituted primary para-phenylenediamine of formula (I) may be chosen, for example, from 2-isopropyl-6-methylbenzene-1,4-diamine, 2-ethyl-6-methylbenzene-1,4-diamine, 2-tert-butyl-6-methylbenzene-1,4-diamine, 2,5-diamino-3-methylbenzoic acid, 2-methoxy-6-methylbenzene-1,4-diamine, 2-methoxy-6-(2-pyrrolidin-1-ylethyl)benzene-1,4-diamine, 2-(3-dimethylaminopropyl)-6-methylbenzene-1,4-diamine, 2-(3-imidazol-1-ylpropyl)-6-methylbenzene-1,4-diamine, 2,5-diaminoisophthalamide, 2,5-diamino-3-methylbenzamide, 1-(2,5-diamino-3-methylphenyl)ethanol, 2-methyl-6-propylbenzene-1,4-diamine, 2-isobutyl-6-methylbenzene-1,4-diamine, 2-(2,5-diamino-3-methylphenyl)ethanol, and the physiologically acceptable salts and/or solvates thereof.

Further disclosed herein is a process for synthesizing a para-phenylenediamine compound of formula (I) as defined above comprising reducing a compound chosen from corresponding intermediate compounds of formulas (II), (III), and (IV):

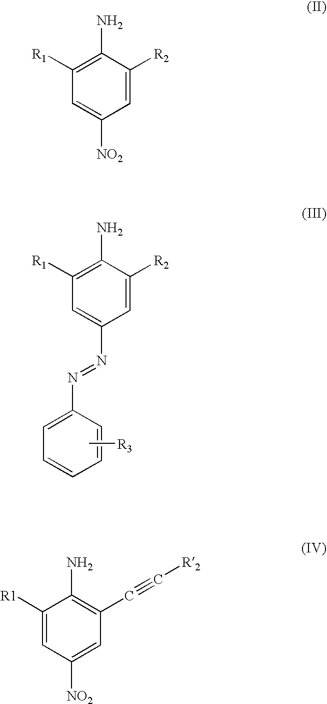

wherein:

R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;

R′₂ is chosen from:
linear or branched C₁-C₄ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups; and
linear or branched C₁-C₆ alkoxy, C₁-C₆ monohydroxyalkoxy, and C₁-C₆ polyhydroxyalkoxy radicals; and

R₃ is chosen from hydrogen and sulfonic groups.

Also disclosed herein are the intermediate compounds of formula (II):

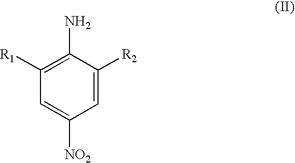

wherein:

R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;

with the proviso that the compounds of formula (II) are not chosen from:

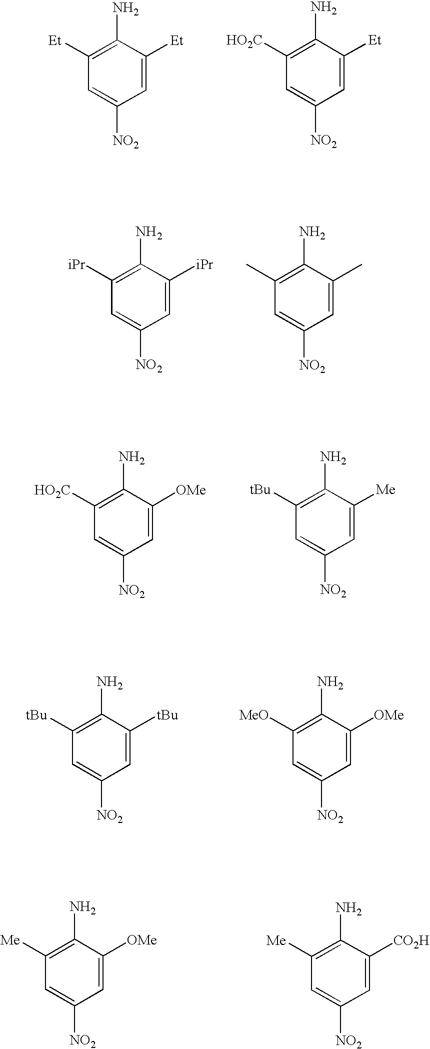

Further disclosed herein are the intermediate compounds of formula (III):

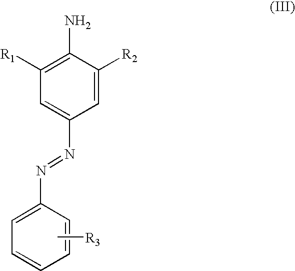

wherein:

R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals;
amide radicals; and

R₃ is chosen from hydrogen and sulfonic groups;

with the proviso that the compounds of formula (III) are not chosen from:

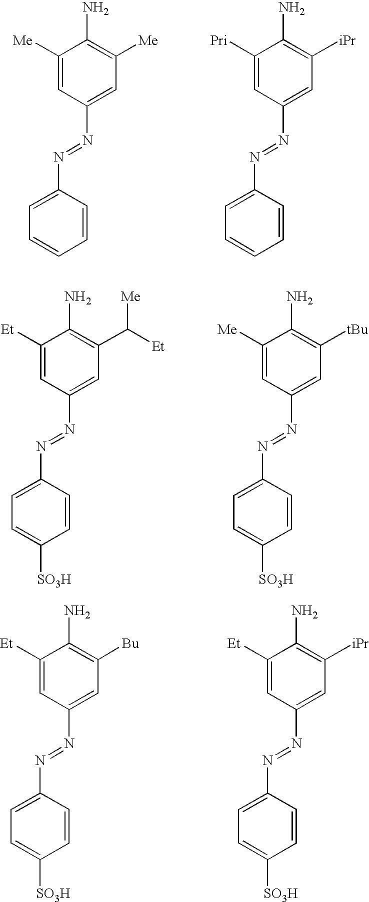

Still further disclosed herein are the intermediate compounds of formula (IV):

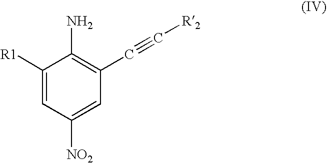

wherein:

R₁ is chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;

and R₁₂ is chosen from:
linear or branched C₁-C₄ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched G₁-C₆ alkoxy, C₁-C₆ monohydroxyalkoxy, and C₁-C₆ polyhydroxyalkoxy radicals;

with the proviso that the compounds of formula (IV) are not chosen from:

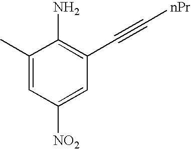

Also disclosed herein is a composition for the oxidation dyeing of keratin fibers, for example, human keratin fibers such as the hair, comprising, in a suitable dyeing medium, at least one para-phenylenediamine compound chosen from compounds of formula (I) and the salts and solvates thereof:

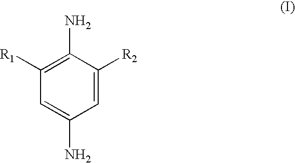

wherein:

R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals such as: 1-pyrrolidinyl, 1-cyclohexylaminyl, 1-piperazinyl, 1-diazepanyl, 1-morpholinyl, and 1-imidazoyl, in which the ring is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radical; and
amide radicals;

with the proviso that the compounds of formula (I) are not chosen from:

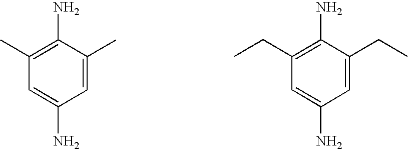

The colorations obtained with the oxidation dye composition in accordance with the present disclosure may be strong. They may also show excellent properties of resistance to the action of various external agents such as light, bad weather, washing, permanent waving, perspiration, and/or rubbing.

Further disclosed herein is a process for the oxidation dyeing of keratin fibers comprising applying a dye composition of the present disclosure to the keratin fibers.

Other characteristics, aspects, subjects, and advantages of the present disclosure will emerge even more clearly upon reading the description and the examples that follow.

Examples of 2,6-substituted primary para-phenylenediamines of formula (I) that may be used in the compositions for oxidation dyeing in accordance with the present disclosure include, but are not limited to, the following compounds and the salts and/or solvates thereof:

| | |
|---|---|
| 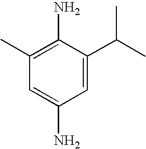 | 2-Isopropyl-6-methylbenzene-1,4-diamine |
| 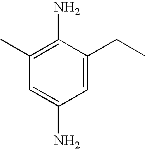 | 2-Ethyl-6-methylbenzene-1,4-diamine |
| 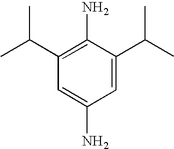 | 2,6-Diisopropylbenzene-1,4-diamine |
| 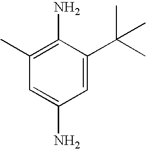 | 2-tert-Butyl-6-methylbenzene-1,4-diamine |
| 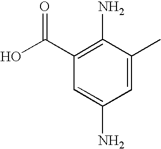 | 2,5-Diamino-3-methylbenzoic acid |
| 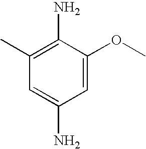 | 2-Methoxy-6-methylbenzene-1,4-diamine |
| 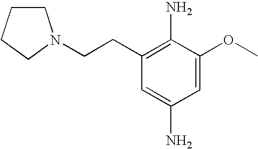 | 2-Methoxy-6-(2-pyrrolidin-1-ylethyl)benzene-1,4- diamine |
| 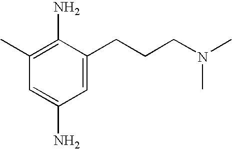 | 2-(3-Dimethylaminopropyl)-6-methylbenzene-1,4- diamine |
| 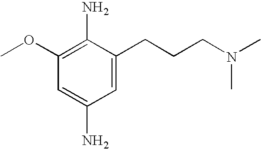 | 2-(3-Dimethylaminopropyl)-6-methoxybenzene- 1,4-diamine |
| 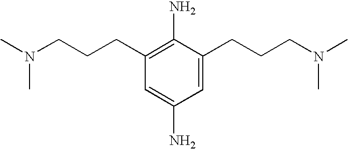 | 2,6-Bis(3-dimethylaminopropyl)benzene-1,4- diamine |
| 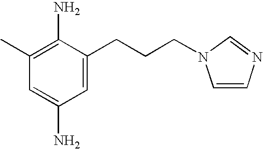 | 2-(3-Imidazol-1-ylpropyl)-6-methylbenzene-1,4- diamine |
| 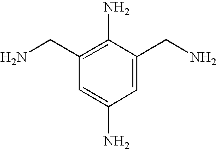 | 2,6-Bisaminomethylbenzene-1,4-diamine |
| 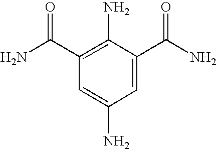 | 2,5-Diaminoisophthalamide |
| 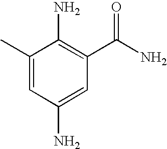 | 2,5-Diamino-3-methylbenzamide |
| 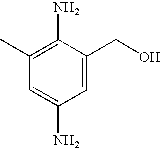 | (2,5-Diamino-3-methylphenyl)methanol |
| 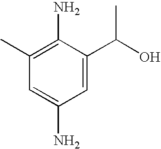 | 1-(2,5-Diamino-3-methylphenyl)ethanol |
| 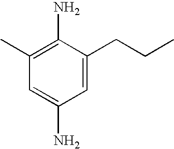 | 2-Methyl-6-propylbenzene-1,4-diamine |
| 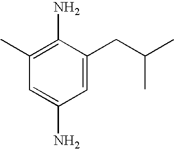 | 2-Isobutyl-6-methylbenzene-1,4-diamine |
| 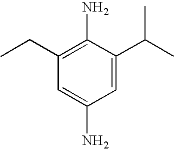 | 2-Ethyl-6-isopropylbenzene-1,4-diamine |
| 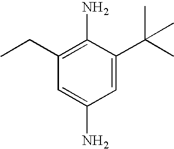 | 2-tert-Butyl-6-ethylbenzene-1,4-diamine |
| 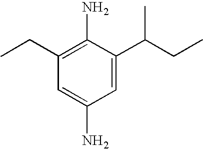 | 2-sec-Butyl-6-ethylbenzene-1,4-diamine |
| 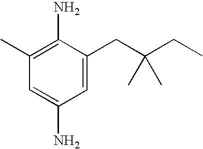 | 2-(2,2-Dimethylbutyl)-6-methylbenzene-1,4- diamine |
| 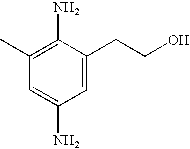 | 2-(2,5-Diamino-3-methylphenyl)ethanol |
| 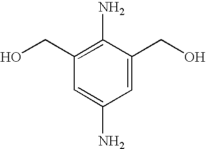 | (2,5-Diamino-3-hydroxymethylphenyl)methanol |
| 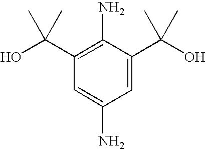 | 2-[2,5-Diamino-3-(1-hydroxy-1-methyl- ethyl)phenyl]propan-2-ol |
| 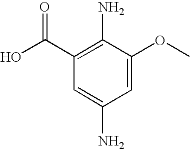 | 2,5-Diamino-3-methoxybenzoic acid |
| 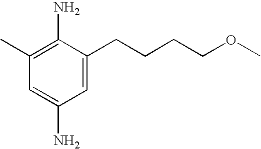 | 2-(4-Methoxybutyl)-6-methylbenzene-1,4-diamine |

In at least one embodiment, the 2,6-substituted primary para-phenylenediamines of formula (I) that may be used in the compositions for oxidation dyeing in accordance with the present disclosure may be chosen, for example, from:

2-isopropyl-6-methylbenzene-1,4-diamine, 2-ethyl-6-methylbenzene-1,4-diamine, 2-tert-butyl-6-methylbenzene-1,4-diamine, 2,5-diamino-3-methylbenzoic acid, 2-methoxy-6-methylbenzene-1,4-diamine, 2-methoxy-6-(2-pyrrolidin-1-ylethyl)benzene-1,4-diamine, 2-(3-dimethylaminopropyl)-6-methylbenzene-1,4-diamine, 2-(3-imidazol-1-ylpropyl)-6-methylbenzene-1,4-diamine, 2,5-diaminoisophthalamide, 2,5-diamino-3-methylbenzamide, 1-(2,5-diamino-3-methylphenyl)ethanol, 2-methyl-6-propylbenzene-1,4-diamine, 2-isobutyl-6-methylbenzene-1,4-diamine, 2-(2,5-diamino-3-methylphenyl)ethanol, and physiologically acceptable salts and solvates thereof.

The 2,6-substituted primary para-phenylenediamines of formula (I) may be prepared according to various synthetic routes, for example, according to the three synthetic routes below:

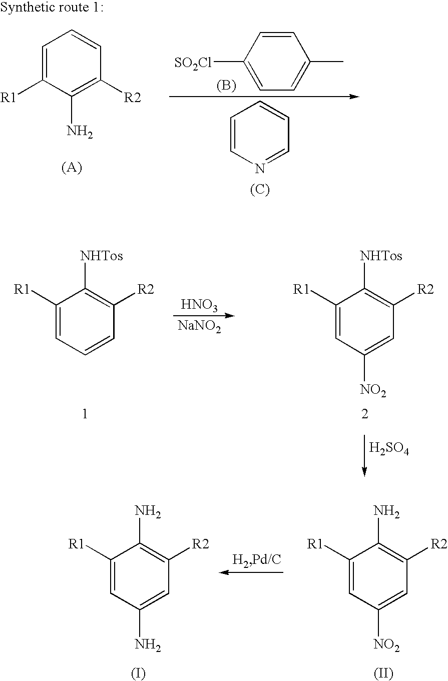

The steps of protecting the amine with a tosyl group, of nitration and of deprotection of the amine were performed according to the method described, for example, in Rec. Trav. Chim., 73: 809-818 (1954). The final step to produce the compounds (I) is a standard reduction step, which may be, for example, a hydrogenation reaction under heterogeneous catalysis in the presence of Pd/C, or alternatively, a reduction reaction with sodium bisulfite, with certain metals (for example, zinc) or with boranes (see, e.g., J., March, Advanced Organic Chemistry, 4th edition, Wiley Interscience, (1992) and M. Hudlicky, Reduction in Organic Chemistry, Ellis Honwood series Chemical Science (1983)).

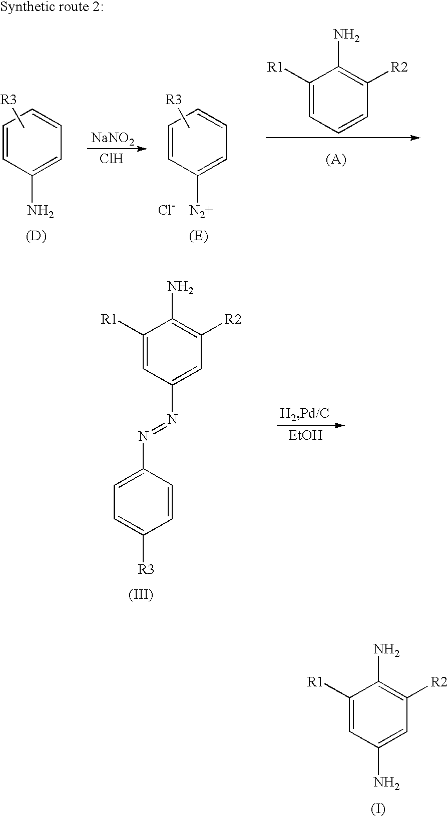

The first step of the synthesis is the formation of an azo compound (III) by reaction of a diazonium salt (E) with an aniline (A) according to well-known methods (see, e.g., Hegarty, in S. Patai, The Chemistry of Diazonium and Diazo Group, pt. 2; Wiley: New York (1978)), the diazonium salt (E) being formed from the aniline (D). The second step to produce the compounds (I) is a standard reduction step, which may be, for example, a hydrogenation reaction under heterogeneous catalysis in the presence of Pd/C, or alternatively, a reduction reaction with sodium bisulfite, with certain metals (fo example, zinc) or with boranes (J. March, Advanced Organic Chemistry, 4th edition, Wiley Interscience (1992) and M. Hudlicky, Reduction in Organic Chemistry, Ellis Honwood series Chemical Science (1983)).

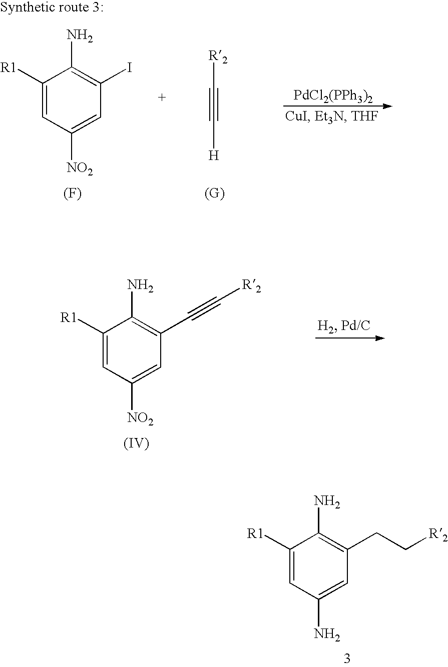

The first step of the synthesis is a Sonogashira reaction between compound (F) and compound (G), described, for instance, in J. Org. Chem. 68: 3327-3329 (2003). The second step to produce the compounds 3 is a standard reduction step, which may be, for example, a hydrogenation reaction under heterogeneous catalysis in the presence of Pd/C, or alternatively, a reduction reaction with sodium bisulfite, with certain metals (for example, zinc) or with boranes (J. March, Advanced Organic Chemistry, 4th edition, Wiley Interscience (1992) and M. Hudlicky, Reduction in Organic Chemistry, Ellis Honwood series, Chemical Science (1983)).

In synthetic routes 1-3, R₁, R₂, R′₂, and R₃ have the meanings given above.

As used herein, the term “corresponding intermediate compound of formula (II), (III), or (IV)” means that the radical R₁ of the intermediate compound of formula (II), (III), or (IV) has the same meaning as that of compound (I). This is likewise the case for R₂ in the intermediate compounds of formula (II) or (III).

In at least one embodiment, an intermediate compound of formula (III) bearing the alkyne group —CC—R₁₂ is reduced to produce the group —CH₂—CH₂—R′₂.

The dye composition according to the present disclosure may comprise from 0.001% to 10% by weight, for example, from 0.05% to 6% by weight, or from 0.1% to 3% by weight, of at least one 2,6-substituted primary para-phenylenediamine chosen from compounds of formula (I) and the salts and solvates thereof.

The dye composition in accordance with the present disclosure may also comprise, in addition to the at least one 2,6-substituted primary para-phenylenediamine, at least one additional oxidation base that may be chosen from oxidation bases conventionally used in oxidation dyeing, for example, additional para-phenylenediamines other than the 2,6-substituted primary para-phenylenes of formula (I), bisphenylalkylenediamines, para-aminophenols, ortho-aminophenols, and heterocyclic bases.

Examples of additional para-phenylenediamines include, but are not limited to, para-phenylenediamine, para-tolylenediamine, 2,6-dimethyl para-phenylenediamine, 2-β-hydroxyethyl-para-phenylenediamine, N-dipropyl-para-phenylenediam ine, 2-isopropyl-para-phenylenediamine, N-(β-hydroxypropyl)-para-phenylenediamine, N,N-bis(β-hydroxyethyl)-para-phenylenediamine, 4-amino-N-(β-methoxyethyl)aniline, the para-phenylenediamines described in French Patent Application No. 2 630 438, and the addition salts thereof.

Suitable bis(phenyl)alkylenediamines may be chosen, for example, from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetra-methylenediamine, N,N′-bis(β-hydroxyethyl)-N,N′-bis(4-amino-phenyl)tetramethylenediam ine, N,N′-bis(4-methylaminophenyl)tetra-methylenediamine, N,N′-bis(ethyl)-N,N′-bis(4′-amino-3′-methylphenyl)ethylenediamine, and the addition salts thereof.

Non-limiting examples of para-aminophenols include para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and the addition salts thereof.

In one embodiment, the ortho-aminophenols may be chosen from 2-aminophenol, 2-amino-5-methylphenol, 2-amino-6-methylphenol, 5-acetamido-2-aminophenol, 5-[(2-hydroxyethyl)amino]-2-methylphenol, and the addition salts thereof.

Examples of heterocyclic bases include, but are not limited to, pyridine derivatives, pyrimidine derivatives and pyrazole derivatives, and the addition salts thereof.

The at least one additional oxidation base may be present in the composition in an amount ranging from 0.0005% to 12% by weight relative to the total weight of the dye composition, for example, from 0.005% to 6% by weight relative to the total weight of the dye composition.

The oxidation dye compositions in accordance with the present disclosure may also comprise at least one coupler and/or at least one direct dye, for example, to modify the shades or to enrich them with tints.

The couplers that may be used in the oxidation dye compositions in accordance with the present disclosure may be chosen, for example, from the couplers conventionally used in oxidation dyeing, such as meta-phenylenediamines, meta-aminophenols, meta-diphenols, monohydroxylated or polyhydroxylated naphthalene derivatives, and heterocyclic couplers, for instance, indole and pyridine derivatives, and the addition salts thereof.

In at least one embodiment, these couplers may be chosen from 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

The at least one coupler may be present in the dye composition in an amount ranging from 0.0001% to 10%, for example, from 0.005% to 5% by weight, or from 0.1% to 3% by weight relative to the total weight of the dye composition.

In general, the acid-addition salts that may be used in the context of the dye compositions of the present disclosure (oxidation bases and couplers) may be chosen, for example, from the salts of hydrochloric acid, hydrobromic acid, sulfuric acid, acetic acid, lactic acid, tartaric acid, citric acid, methanesulfonic acid, and succinic acid.

The suitable dyeing medium (or support) useful in accordance with the present disclosure may be chosen from water and mixtures of water and at least one organic solvent chosen, for instance, from C₁-C₄ lower alkanols, polyols, polyol ethers, aromatic alcohols, and mixtures thereof.

The dye composition according to the present disclosure may also comprise at least one of various adjuvants conventionally used in hair-dye compositions, such as anionic, cationic, nonionic, amphoteric, or zwitterionic surfactants and mixtures thereof; anionic, cationic, nonionic, amphoteric, or zwitterionic polymers and mixtures thereof; mineral and organic thickeners; antioxidants; reducing agents; sunscreens; penetrants; sequestrants; fragrances; buffers; dispersants; and conditioning agents, for instance, silicones, film-forming agents, preserving agents, and opacifiers.

In at least one embodiment, the pH of the dye composition according to the present disclosure ranges from 3 to 12.

It is to be understood that a person skilled in the art will take care to select the at least one optional additional compound such that the advantageous properties intrinsically associated with the oxidation dye composition in accordance with the present disclosure are not, or are not substantially, adversely affected by the envisaged addition.

The dye composition according to the present disclosure may be in various forms, such as liquids, creams, and gels, or any other form that is suitable for dyeing keratin fibers, for example, human hair.

Also disclosed herein is a process for dyeing keratin fibers, for example, human keratin fibers, such as the hair, comprising applying at least one dye composition of the present disclosure to the keratin fibers.

According to one embodiment, at least one dye composition of the present disclosure is applied to the fibers for a time that is sufficient to develop the desired coloration, either in air or using an oxidizing agent. In another embodiment, the dye composition may optionally comprise at least one oxidation catalyst in order to accelerate the oxidation process.

According to yet another embodiment of the process of the present disclosure, the coloration of the fibers may be performed without addition of an oxidizing agent, merely on contact with atmospheric oxygen.

According to a further embodiment of the process of the present disclosure, at least one dye composition according to the present disclosure is applied to the fibers, and the color is revealed at a pH chosen from acidic, neutral, and alkaline pHs using at least one oxidizing agent that is added just at the time of application to the dye composition or that is present in an oxidizing composition applied simultaneously or sequentially in a separate manner.

According to this embodiment, the dye composition described above may be mixed, at the time of use, with an oxidizing composition comprising, in a suitable dyeing medium, at least one oxidizing agent present in an amount sufficient to develop a coloration. The mixture obtained is then applied to the keratin fibers and is left to act for 3 to 50 minutes, for example, from 5 to 30 minutes, after which the fibers are rinsed, washed with shampoo, rinsed again, and dried.

The at least one oxidizing agent present in the oxidizing composition as defined above may be chosen from the oxidizing agents conventionally used for the oxidation dyeing of keratin fibers, for instance, hydrogen peroxide, urea peroxide, alkali metal bromates, and persalts such as perborates and persulfates. In one embodiment, the at least one oxidizing agent is hydrogen peroxide.

The pH of the oxidizing composition comprising the at least one oxidizing agent may be such that, after mixing with the dye composition, the pH of the resulting composition applied to the keratin fibers ranges, for example, from 3 to 12, for example, from 5 to 11. The pH may be adjusted to the desired value by means of acidifying or basifying agents conventionally used in the dyeing of keratin fibers and as defined above.

The oxidizing composition as defined above may also contain various adjuvants conventionally used in hair dye compositions and as defined above.

The composition that is finally applied to the keratin fibers may be in various forms, such as liquids, creams, and gels, or any other form that is suitable for dyeing keratin fibers, for example, human hair.

Further disclosed herein is a multi-compartment device or dyeing “kit” or any other multi-compartment packaging system, comprising at least one first compartment containing at least one dye composition of the present disclosure and at least one second compartment containing at least one oxidizing composition as defined above. These devices may be equipped with a means for applying the desired mixture to the hair, such as the devices described in French Patent No. 2 586 913.

Still further disclose herein is the use of the compounds of the present disclosure as oxidation dye precursors.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Synthesis Examples

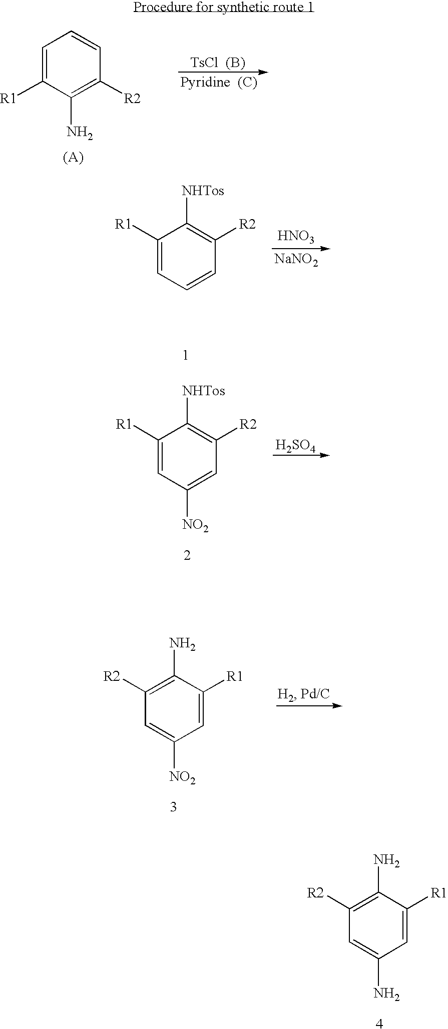

Synthesis of Compounds (1)

The aniline (A), para-toluenesulfonyl chloride (B), and pyridine (C) were refluxed overnight. The reaction mixture was cooled and poured onto ice, and the precipitate corresponding to compounds 1 was filtered off, washed with water, and dried.

### 1a: N-(2-Ethyl-6-methylphenyl)-4-methylbenzenesulfonamide

Aniline (A): 6-Ethyl-o-toluidine: 4.7 g, 1 eq.

Tosyl chloride (B): 7.96 g, 1.2 eq.

Pyridine (C): 7 ml

Mass obtained: 9.38 g (yield: 93%)

Beige-colored solid

M (m/z): M⁻=288

### 1b: N-(2-Isopropyl-6-methylphenyl)-4-methylbenzenesulfonamide

Aniline (A): 2-Isopropyl-6-methylaniline 4.7 g, 1 eq.

Tosyl chloride (B): 7.21 g, 1.2 eq.

Pyridine (C): 7 ml

Mass obtained: 8.17 g (yield: 86%)

White solid

M (m/z): M⁻=302

### 1c: N-(2-tert-Butyl-6-methylphenyl)-4-methylbenzenesulfonamide

Aniline (A): 6-tert-Butyl-o-toluidine: 5 g, 1 eq.

Tosyl chloride (B): 7 g, 1.2 eq.

Pyridine (C): 10 ml

Mass obtained: 9.5 g (yield: 98%)

Beige-colored solid

M (m/z): M⁻=316

Synthesis of Compounds (2)

The tosyl aniline 1, concentrated nitric acid, glacial acetic acid, sodium nitrite, and distilled water were refluxed for 3 hours. The reaction mixture was cooled and poured onto ice. The precipitate corresponding to compounds 2 was filtered off, washed with water, and dried under vacuum.

### 2a: N-(2-Ethyl-6-methyl-4-nitrophenyl)-4-methylbenzenesulfonamide

Reagent 1a: 2 g, 1 eq.

Nitric acid: 1.74 ml, 6 eq.

Sodium nitrite: 0.05 g, 0.1 eq.

Water: 16 ml

Glacial acetic acid: 16 ml

Mass obtained: 1.66 g (72% yield)

Pale yellow solid

M(m/z): M⁻=333

### 2b: N-(2-Isopropyl-6-methyl-4-nitrophenyl)-4-methylbenzenesulfonamide

Reagent 1b: 4 g, 1 eq.

Nitric acid: 3.28 ml, 6 eq.

Sodium nitrite: 0.09 g, 0.1 eq.

Water: 25 ml

Glacial acetic acid: 25 ml

Mass obtained: 5 g (100% yield)

Pale yellow solid

M(m/z): M⁻=347

### 2c: N-(2-tert-Butyl-6-methyl-4-nitrophenyl)-4-methylbenzenesulfonamide

Reagent 1c: 5 g, 1 eq.

Nitric acid: 3.92 ml, 6 eq.

Sodium nitrite: 0.108 g, 0.1 eq.

Water: 30 ml

Glacial acetic acid: 30 ml

Mass obtained: 5.1 g (89% yield)

Beige-colored solid

M(m/z): M⁻=361

Synthesis of the Compounds (3)

Compound 2 was added to a mixture of concentrated sulfuric acid and distilled water at room temperature overnight. The reaction mixture was poured into ice and then basified with sodium hydroxide. The precipitate obtained corresponding to compound 3 was filtered off and washed with water.

### 3a: 2-Ethyl-6-methyl-4-nitroaniline

Reagent 2a: 1.5 g

Concentrated sulfuric acid: 50 ml

Distilled water: 5 ml

Mass obtained: 0.78 g (97% yield)

Orange solid

M(m/z): M⁻=179

### 3b: 2-Isopropyl-6-methyl-4-nitroaniline

Reagent 2b: 5 g

Concentrated sulfuric acid: 50 ml

Distilled water: 5 ml

Mass obtained: 1.0 g (37% yield)

Green solid

M(m/z): M⁻=193

### 3c: 2-tert-Butyl-6-methyl-4-nitroaniline

Reagent 2c: 5 g

Concentrated sulfuric acid: 50 ml

Distilled water: 5 ml

Mass obtained: 2.5 g (87% yield)

Green solid

M(m/z): M⁻=207

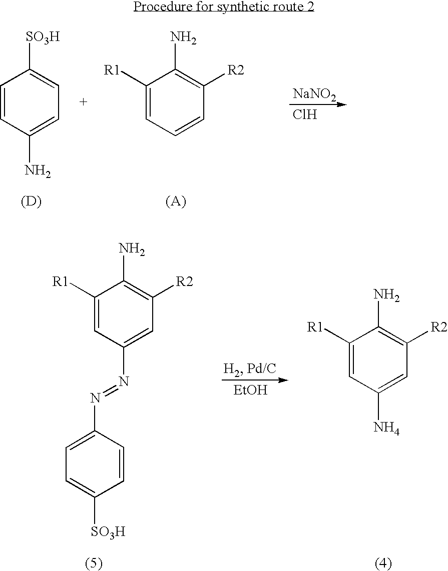

Synthesis of the Compounds (5)

A solution of sodium nitrite in water was added without heating to sulfanilic acid dissolved in water (orange color). The temperature was maintained below 10° C.

The solution obtained was immediately poured into a mixture of concentrated hydrochloric acid and ice.

The cold diazonium salt was added to the aniline (A) dissolved in 0.5 N hydrochloric acid (a blood-orange color appears). The precipitate formed corresponding to the compounds 5 was filtered off and washed once with water.

### 5d: 4-[(E)-(4-Amino-3-methoxy-5-methylphenyl)diazenyl]benzenesulfonic acid

Amine (A): 2-Methoxy-6-methylaniline, 6.85 g, 1 eq.

HCl (0.5 N): 100 ml

Sulfanilic acid: 8.66 g (1 eq.) in 50 ml of water

Sodium nitrite: 3.73 g (1.08 eq.) in 10 ml of water

35% hydrochloric acid: 12.5 ml (2.5 eq.) in 60 g of ice

Mass obtained: 6.1 g (38% yield)

Red solid

M(m/z): M⁻=320

### 5e: 4-{(E)-[4-Amino-3-methoxy-5-(2-pyrrolidin-1-ylethyl)phenyl]-diazenyl}benzenesulfonic acid

Amine (A): 1-(2-(2-amino-3-methoxyphenyl)ethyl)pyrrolidine, 5 g, 1 eq.

HCl (0.5 N): 80 ml

Sulfanilic acid: 3.93 g, 1 eq. in 25 ml of water

Sodium nitrite: 1.69 g, 1.08 eq. in 5 ml of water

35% hydrochloric acid: 6 ml, 2.5 eq. in 30 g of ice

Mass obtained: 2.52 g (27% yield)

Red solid

M(m/z): M⁺=405

Reduction Step of Routes 1 and 2

The compound 3 or 5, catalyst (palladium-on-charcoal) and ethanol were placed in a 0.5-litre hydrogenator.

The reduction was performed under a hydrogen pressure of about six bar at a temperature of 50° C. After filtering off the catalyst under nitrogen, the filtrate was poured into aqueous hydrochloric acid. The filtrate was evaporated to dryness under reduced pressure. The product was crystallized from hydrochloric ethanol and dried at 40° C. under vacuum and over potassium hydroxide.

### 4a: 2-Ethyl-6-methylbenzene-1,4-diamine dihydrochloride

Reagent 3a: 0.8 g

Pd/C, 0.2 g

Absolute ethanol: 250 ml

Mass obtained: 0.6 g (91% yield)

Beige-colored solid

M(m/z): M⁺=151

### 4b: 2-Isopropyl-6-methylbenzene-1,4-diamine dihydrochloride

Reagent 3b: 1 g

Pd/C, 0.3 g

Absolute ethanol: 250 ml

Mass obtained: 0.82 g (97% yield)

Beige-colored solid

M(m/z): M⁺=165

### 4c: 2-tert-Butyl-6-methylbenzene-1,4-diamine dihydrochloride

Reagent 3c: 2.2 g

Pd/C, 0.6 g

Absolute ethanol: 250 ml

Mass obtained: 2.15 g (81% yield)

Beige-colored solid

M(m/z): M⁺=1 79

### 4d: 2-Methoxy-6-methylbenzene-1,4-diamine dihydrochloride

Reagent 5d: 6.1 g

Pd/C, 1.2 g

Absolute ethanol: 250 ml

Mass obtained: 2.91 g (68% yield)

Pink solid

M(m/z): M⁺=153

### 4e: 2-Methoxy-6-(2-pyrrolidin-1-ylethyl)benzene-1,4-diamine dihydrochloride

Reagent 5e: 2.45 g

Pd/C, 0.8 g

Absolute ethanol: 250 ml

Mass obtained: 0.75 g (40% yield)

Green solid

M(m/z): M⁺=236

### 4f: 2,5-Diamino-3-methylbenzoic acid dihydrochloride

Commercial reagent: 2-amino-3-methyl-5-nitrobenzoic acid: 2 g

Pd/C, 0.2 g

Absolute ethanol: 250 ml

Mass obtained: 1.3 g (70% yield)

Beige-colored solid

M(m/z): M⁺=165

### 4g: 2,6-Diisopropylbenzene-1,4-diamine dihydrochloride

Commercial reagent: 2,6-Diisopropyl-4-nitrophenylamine: 2 g

Pd/C, 0.3 g

Absolute ethanol: 250 ml

Mass obtained: 1.68 g (66% yield)

Beige-colored solid

M(m/z): M⁺=193

### Formulation Examples

### Examples 1 to 5

### Dye composition using 2,5-diamino-3-methylbenzoic acid dihydrochloride (4f)

### Examples 1 to 3: Dyeing in Acidic Medium

The dye compositions below were prepared:

| | Example | | | | | |
|---|---|---|---|---|---|---|
| | 1 | | 2 | | 3 | |
| 2,5-Diamino-3-methyl- | 10⁻³ | mol | 10⁻³ | mol | 10⁻³ | mol |
| benzoic acid dihydrochloride | | | | | | |
| 2-Aminopyridin-3-ol | 10⁻³ | mol | | | | |
| 2-(2,4-Diaminophenoxy) | | | 10⁻³ | mol | | |
| ethanol hydrochloride | | | | | | |
| 3-Amino-2-chloro-6- | | | | | 10⁻³ | mol |
| methylphenol hydrochloride | | | | | | |
| Dye support (1) | (*) | | (*) | | (*) | |
| Demineralized water qs | 100 | g | 100 | g | 100 | g |

(*): dye support (1) pH 7

| | |
|---|---|
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair containing 90% white hairs. After a leave-on time of 30 minutes, the locks were rinsed, washed with a standard shampoo, rinsed again, and then dried.

The shades obtained are given in the table below:

| | Example | | |
|---|---|---|---|
| | 1 | 2 | 3 |
| Shade observed | orange | blue-grey | red-violet |
| | | | grey |

### Examples 4 to 5

### Dyeing in Basic Medium

The dye compositions below were prepared:

| | Example | | | |
|---|---|---|---|---|
| | 4 | | 5 | |
| 2,5-Diamino-3-methylbenzoic | 10⁻³ | mol | 10⁻³ | mol |
| acid dihydrochloride | | | | |
| 2-(2,4-Diaminophenoxy)ethanol | 10⁻³ | mol | | |
| hydrochloride | | | | |
| 3-Amino-2-chloro-6- | | | 10⁻³ | mol |
| methylphenol hydrochloride | | | | |
| Dye support (2) | (*) | | (*) | |
| Demineralized water qs | 100 | g | 100 | g |

(*): dye support (2) pH 9.5

| | |
|---|---|
| 96° Ethyl alcohol | 20.8 g |
| Sodium metabisulfite as an aqueous 35% solution | 0.23 g AM |
| Pentasodium salt of diethylenetriaminepentaacetic acid | 0.48 g AM |
| as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

Example

| | 4 | 5 |
|---|---|---|
| Shade observed | blue-grey | red-violet |

### Examples 6 to 18

### Dye composition using 2,6-diisopropylbenzene-1,4-diamine dihydrochloride (4 g)

### Examples 6 to 12

### Dyeing in Acidic Medium

The dye compositions below were prepared:

| | Example | | | | | | |
|---|---|---|---|---|---|---|---|
| | 6 | 7 | 8 | 9 | 10 | 11 | 12 |
| 2,6-Diisopropylbenzene- | 10⁻³ mol | 10⁻³ mol | 10⁻³ mol | 10⁻³ mol | 10⁻³ mol | 10⁻³ mol | 10⁻³ mol |
| 1,4- | | | | | | | |
| diamine | | | | | | | |
| dihydrochloride | | | | | | | |
| Benzene-1,3- | 10⁻³ mol | | | | | | |
| diol | | | | | | | |
| 5-Amino-2- | | 10⁻³ mol | | | | | |
| methylphenol | | | | | | | |
| 1H-Indol-6-ol | | | 10⁻³ mol | | | | |
| 2-Aminopyridin- | | | | 10⁻³ mol | | | |
| 3-ol | | | | | | | |
| 3,6-Dimethyl- | | | | | 10⁻³ mol | | |
| 1H- | | | | | | | |
| pyrazolo[5,1- | | | | | | | |
| c][1,2,4]triazole | | | | | | | |
| 2-(2,4-Diamino- | | | | | | 10⁻³ mol | |
| phenoxy)ethanol | | | | | | | |
| hydrochloride | | | | | | | |
| 3-Amino-2- | | | | | | | 10⁻³ mol |
| chloro-6- | | | | | | | |
| methylphenol | | | | | | | |
| hydrochloride | | | | | | | |
| Dye support (1) | (*) | (*) | (*) | (*) | (*) | (*) | (*) |
| Demineralized | 100 g | 100 g | 100 g | 100 g | 100 g | 100 g | 100 g |
| water qs | | | | | | | |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair containing 90% white hairs. After a leave-on time of 30 minutes, the locks were rinsed, washed with a standard shampoo, rinsed again, and then dried.

The shades obtained are given in the table below:

| | Example | | | | | | |
|---|---|---|---|---|---|---|---|
| | 6 | 7 | 8 | 9 | 10 | 11 | 12 |
| Shade | yellow | strong | strong | strong | red | strong | strong |
| observed | | red | red | red | | blue | violet |

### Examples 13 to 18

### Dyeing in Basic Medium

The dye compositions below were prepared:

| | Example | | | | | |
|---|---|---|---|---|---|---|
| | 13 | 14 | 15 | 16 | 17 | 18 |
| 2,6-Diisopropylbenzene- | 10⁻³ mol | 10⁻³ mol | 10⁻³ mol | 10⁻³ mol | 10⁻³ mol | 10⁻³ mol |
| 1,4- | | | | | | |
| diamine | | | | | | |
| dihydrochloride | | | | | | |
| 5-Amino-2- | 10⁻³ mol | | | | | |
| methylphenol | | | | | | |
| 1H-Indol-6-ol | | 10⁻³ mol | | | | |
| 2-Aminopyridin- | | | 10⁻³ mol | | | |
| 3-ol | | | | | | |
| 3,6-Dimethyl-1H- | | | | 10⁻³ mol | | |
| pyrazolo[5,1- | | | | | | |
| c][1,2,4]triazole | | | | | | |
| 2-(2,4-Diamino- | | | | | 10⁻³ mol | |
| phenoxy)ethanol | | | | | | |
| hydrochloride | | | | | | |
| 3-Amino-2- | | | | | | 10⁻³ mol |
| chloro-6- | | | | | | |
| methylphenol | | | | | | |
| hydrochloride | | | | | | |
| Dye support (2) | (*) | (*) | (*) | (*) | (*) | (*) |
| Demineralized | 100 g | 100 g | 100 g | 100 g | 100 g | 100 g |
| water qs | | | | | | |

| | |
|---|---|
| (*): dye support (2) pH 9.5 | |
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | |
|---|---|---|---|---|---|---|
| | 13 | 14 | 15 | 16 | 17 | 18 |
| Shade observed | Red | red | red | chromatic | strong | strong |
| | | | | red | blue | violet |

### Examples 19 to 31

### Dye composition using 2-ethyl-6-methylbenzene-1,4-diamine dihydrochloride (4a)

### Examples 19 to 25

### Dyeing in Acidic Medium

The dye compositions below were prepared:

| | Example | | | | | | |
|---|---|---|---|---|---|---|---|
| | 19 | 20 | 21 | 22 | 23 | 24 | 25 |
| 2-Ethyl-6- | 10⁻³ mol | 10⁻³ mol | 10⁻³ mol | 10⁻³ mol | 10⁻³ mol | 10⁻³ mol | 10⁻³ mol |
| methylbenzene- | | | | | | | |
| 1,4-diamine | | | | | | | |
| dihydrochloride | | | | | | | |
| Benzene-1,3- | 10⁻³ mol | | | | | | |
| diol | | | | | | | |
| 5-Amino-2- | | 10⁻³ mol | | | | | |
| methylphenol | | | | | | | |
| 1H-Indol-6-ol | | | 10⁻³ mol | | | | |
| 2-Aminopyridin- | | | | 10⁻³ mol | | | |
| 3-ol | | | | | | | |
| 3,6-Dimethyl- | | | | | 10⁻³ mol | | |
| 1H- | | | | | | | |
| pyrazolo[5,1- | | | | | | | |
| c][1,2,4]triazole | | | | | | | |
| 2-(2,4-Diamino- | | | | | | 10⁻³ mol | |
| phenoxy)ethanol | | | | | | | |
| hydrochloride | | | | | | | |
| 3-Amino-2- | | | | | | | 10⁻³ mol |
| chloro-6- | | | | | | | |
| methylphenol | | | | | | | |
| hydrochloride | | | | | | | |
| Dye support (1) | (*) | (*) | (*) | (*) | (*) | (*) | (*) |
| Demineralized | 100 g | 100 g | 100 g | 100 g | 100 g | 100 g | 100 g |
| water qs | | | | | | | |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | | |
|---|---|---|---|---|---|---|---|
| | 19 | 20 | 21 | 22 | 23 | 24 | 25 |
| Shade | yellow- | strong | strong | strong | red | strong | strong |
| observed | brown | red- | red | red- | | blue- | violet |
| | | violet | | brown | | violet | |

### Examples 26 to 31

### Dyeing in Basic Medium

The dye compositions below were prepared:

| | Example | | | | | |
|---|---|---|---|---|---|---|
| | 26 | 27 | 28 | 29 | 30 | 31 |
| 2-ethyl-6-methylbenzene- | 10⁻³ mol | 10⁻³ mol | 10⁻³ mol | 10⁻³ mol | 10⁻³ mol | 10⁻³ mol |
| 1,4- | | | | | | |
| diamine | | | | | | |
| dihydrochloride | | | | | | |
| 5-Amino-2- | 10⁻³ mol | | | | | |
| methylphenol | | | | | | |
| 1H-Indol-6-ol | | 10⁻³ mol | | | | |
| 2-Aminopyridin-3-ol | | | 10⁻³ mol | | | |
| 3,6-Dimethyl-1H- | | | | 10⁻³ mol | | |
| pyrazolo[5,1- | | | | | | |
| c][1,2,4]triazole | | | | | | |
| 2-(2,4-Diamino- | | | | | 10⁻³ mol | |
| phenoxy)ethanol | | | | | | |
| hydrochloride | | | | | | |
| 3-Amino-2-chloro- | | | | | | 10⁻³ mol |
| 6-methylphenol | | | | | | |
| hydrochloride | | | | | | |
| Dye support (2) | (*) | (*) | (*) | (*) | (*) | (*) |
| Demineralized | 100 g | 100 g | 100 g | 100 g | 100 g | 100 g |
| water qs | | | | | | |

| | |
|---|---|
| (*): dye support (2) pH 9.5 | |
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | |
|---|---|---|---|---|---|---|
| | 26 | 27 | 28 | 29 | 30 | 31 |
| Shade observed | red | orange | red | chromatic | strong | strong |
| | | | | red | blue | violet |

### Examples 32 to 44

### Dye composition using 2-isopropyl-6-methylbenzene-1,4-diamine dihydrochloride (4b)

### Examples 32 to 38

### Dyeing in Acidic Medium

The dye compositions below were prepared:

| | Example | | | | | | |
|---|---|---|---|---|---|---|---|
| | 32 | 33 | 34 | 35 | 36 | 37 | 38 |
| 2-Isopropyl-6- | 10⁻³ mol | 10⁻³ mol | 10⁻³ mol | 10⁻³ mol | 10⁻³ mol | 10⁻³ mol | 10⁻³ mol |
| methylbenzene- | | | | | | | |
| 1,4-diamine | | | | | | | |
| dihydrochloride | | | | | | | |
| Benzene-1,3-diol | 10⁻³ mol | | | | | | |
| 5-Amino-2- | | 10⁻³ mol | | | | | |
| methylphenol | | | | | | | |
| 1H-Indol-6-ol | | | 10⁻³ mol | | | | |
| 2-Aminopyridin-3-ol | | | | 10⁻³ mol | | | |
| 3,6-Dimethyl-1H- | | | | | 10⁻³ mol | | |
| pyrazolo[5,1- | | | | | | | |
| c][1,2,4]triazole | | | | | | | |
| 2-(2,4-Diamino- | | | | | | 10⁻³ mol | |
| phenoxy)ethanol | | | | | | | |
| hydrochloride | | | | | | | |
| 3-Amino-2-chloro- | | | | | | | 10⁻³ mol |
| 6-methylphenol | | | | | | | |
| hydrochloride | | | | | | | |
| Dye support (1) | (*) | (*) | (*) | (*) | (*) | (*) | (*) |
| Demineralized | 100 g | 100 g | 100 g | 100 g | 100 g | 100 g | 100 g |
| water qs | | | | | | | |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | | |
|---|---|---|---|---|---|---|---|
| | 32 | 33 | 34 | 35 | 36 | 37 | 38 |
| Shade | yellow- | strong | orange | strong | red | strong | strong |
| observed | brown | red | | red- | | blue | violet |
| | | | | brown | | | |

### Examples 39 to 44

### Dyeing in Basic Medium

The dye compositions below were prepared:

| | Example | | | | | |
|---|---|---|---|---|---|---|
| | 39 | 40 | 41 | 42 | 43 | 44 |
| 2-Isopropyl-6- | 10⁻³ mol | 10⁻³ mol | 10⁻³ mol | 10⁻³ mol | 10⁻³ mol | 10⁻³ mol |
| methylbenzene-1,4- | | | | | | |
| diamine | | | | | | |
| dihydrochloride | | | | | | |
| 5-Amino-2- | 10⁻³ mol | | | | | |
| methylphenol | | | | | | |
| 1H-Indol-6-ol | | 10⁻³ mol | | | | |
| 2-Aminopyridin-3-ol | | | 10⁻³ mol | | | |
| 3,6-Dimethyl-1H- | | | | 10⁻³ mol | | |
| pyrazolo[5,1- | | | | | | |
| c][1,2,4]triazole | | | | | | |
| 2-(2,4-Diaminophenoxy) | | | | | 10⁻³ mol | |
| ethanol | | | | | | |
| hydrochloride | | | | | | |
| 3-Amino-2-chloro-6- | | | | | | 10⁻³ mol |
| methylphenol | | | | | | |
| hydrochloride | | | | | | |
| Dye support (2) | (*) | (*) | (*) | (*) | (*) | (*) |
| Demineralized | 100 g | 100 g | 100 g | 100 g | 100 g | 100 g |
| water qs | | | | | | |

| | |
|---|---|
| (*): dye support (2) pH 9.5 | |
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | |
|---|---|---|---|---|---|---|
| | 39 | 40 | 41 | 42 | 43 | 44 |
| Shade observed | red | orange | red | chromatic | strong | strong |
| | | | | red | blue | violet |

### Examples 45 to 57

### Dye composition using 2-tert-butyl-6-methylbenzene-1,4-diamine dihydrochloride (4c)

### Examples 45 to 51

### Dyeing in Acidic Medium

The dye compositions below were prepared:

| | Example | | | | | | |
|---|---|---|---|---|---|---|---|
| | 45 | 46 | 47 | 48 | 49 | 50 | 51 |
| 2-tert-Butyl-6- | 10⁻³ mol | 10⁻³ mol | 10⁻³ mol | 10⁻³ mol | 10⁻³ mol | 10⁻³ mol | 10⁻³ mol |
| methylbenzene- | | | | | | | |
| 1,4-diamine | | | | | | | |
| dihydrochloride | | | | | | | |
| Benzene-1,3-diol | 10⁻³ mol | | | | | | |
| 5-Amino-2- | | 10⁻³ mol | | | | | |
| methyl-phenol | | | | | | | |
| 1H-Indol-6-ol | | | 10⁻³ mol | | | | |
| 2-Aminopyridin- | | | | 10⁻³ mol | | | |
| 3-ol | | | | | | | |
| 3,6-Dimethyl-1H- | | | | | 10⁻³ mol | | |
| pyrazolo[5,1- | | | | | | | |
| c][1,2,4]triazole | | | | | | | |
| 2-(2,4-Diaminophenoxy) | | | | | | 10⁻³ mol | |
| ethanol | | | | | | | |
| hydrochloride | | | | | | | |
| 3-Amino-2- | | | | | | | 10⁻³ mol |
| chloro-6- | | | | | | | |
| methylphenol | | | | | | | |
| hydrochloride | | | | | | | |
| Dye support (1) | (*) | (*) | (*) | (*) | (*) | (*) | (*) |
| Demineralized | 100 g | 100 g | 100 g | 100 g | 100 g | 100 g | 100 g |
| water qs | | | | | | | |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | | |
|---|---|---|---|---|---|---|---|
| | 45 | 46 | 47 | 48 | 49 | 50 | 51 |
| Shade | orange- | strong | strong | strong | red | strong | strong |
| observed | brown | red- | red | red | | blue | violet |
| | | violet | | | | | |

### Examples 52 to 57

### Dyeing in Basic Medium

The dye compositions below were prepared:

| | Example | | | | | |
|---|---|---|---|---|---|---|
| | 52 | 53 | 54 | 55 | 56 | 57 |
| 2-tert-Butyl-6- | 10⁻³ mol | 10⁻³ mol | 10⁻³ mol | 10⁻³ mol | 10⁻³ mol | 10⁻³ mol |
| methylbenzene-1,4- | | | | | | |
| diamine dihydrochloride | | | | | | |
| 5-Amino-2-methylphenol | 10⁻³ mol | | | | | |
| 1H-Indol-6-ol | | 10⁻³ mol | | | | |
| 2-Amino-pyridin-3-ol | | | 10⁻³ mol | | | |
| 3,6-Dimethyl-1H- | | | | 10⁻³ mol | | |
| pyrazolo[5,1- | | | | | | |
| c][1,2,4]triazole | | | | | | |
| 2-(2,4-Diaminophenoxy) | | | | | 10⁻³ mol | |
| ethanol | | | | | | |
| hydrochloride | | | | | | |
| 3-Amino-2-chloro-6- | | | | | | 10⁻³ mol |
| methylphenol | | | | | | |
| hydrochloride | | | | | | |
| Dye support (2) | (*) | (*) | (*) | (*) | (*) | (*) |
| Demineralized water qs | 100 g | 100 g | 100 g | 100 g | 100 g | 100 g |

| | |
|---|---|
| (*): dye support (2) pH 9.5 | |
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | | |
|---|---|---|---|---|---|---|
| | 52 | 53 | 54 | 55 | 56 | 57 |
| Shade observed | red | red | red | chromatic | strong | strong |
| | | | | red | blue | violet |

### Examples 58 to 64

### Dye composition using 2-methoxy-6-methylbenzene-1,4-diamine dihydrochloride (4d)

### Examples 58 to 62

### Dyeing in Acidic Medium

The dye compositions below were prepared:

| | Examples | | | | |
|---|---|---|---|---|---|
| | 58 | 59 | 60 | 61 | 62 |
| 2-Methoxy-6- | 10⁻³ mol | 10⁻³ mol | 10⁻³ mol | 10⁻³ mol | 10⁻³ mol |
| methylbenzene-1,4-diamine | | | | | |
| dihydrochloride | | | | | |
| 5-Amino-2-methylphenol | 10⁻³ mol | | | | |
| 1H-Indol-6-ol | | 10⁻³ mol | | | |
| 2-Aminopyridin-3-ol | | | 10⁻³ mol | | |
| 2-(2,4- | | | | 10⁻³ mol | |
| Diaminophenoxy)ethanol | | | | | |
| hydrochloride | | | | | |
| 3-Amino-2-chloro-6- | | | | | 10⁻³ mol |
| methylphenol | | | | | |
| hydrochloride | | | | | |
| Dye support (2) | (*) | (*) | (*) | (*) | (*) |
| Demineralized water qs | 100 g | 100 g | 100 g | 100 g | 100 g |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | | | |
|---|---|---|---|---|---|
| | 58 | 59 | 60 | 61 | 62 |
| Shade | red- | strong red | red-brown | strong | strong blue- |
| observed | brown | | | blue-grey | violet grey |

### Examples 63 to 64

### Dyeing in Basic Medium

The dye compositions below were prepared:

| | Example | | | |
|---|---|---|---|---|
| | 63 | | 64 | |
| 2-methoxy-6-methylbenzene-1,4-diamine | 10⁻³ | mol | 10⁻³ | mol |
| dihydrochloride | | | | |
| 5-Amino-2-methylphenol | 10⁻³ | mol | | |
| 3-Amino-2-chloro-6-methylphenol | | | 10⁻³ | mol |
| hydrochloride | | | | |
| Dye support (1) | (*) | | (*) | |
| Demineralized water qs | 100 | g | 100 | g |

(*): dye support (2) pH 9.5

| | |
|---|---|
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

Example

| | 63 | 64 |
|---|---|---|
| Shade observed | blue-grey | blue-violet grey |

### Examples 65 to 72

### Dye composition using 2-methoxy-6-(2-pyrrolidin-1-ylethyl)benzene-1,4-diamine dihydrochloride (4e)

### Examples 65 to 69

### Dyeing in Acidic Medium

The dye compositions below were prepared:

| | Example | | | | |
|---|---|---|---|---|---|
| | 65 | 66 | 67 | 68 | 69 |
| 2-Methoxy-6-(2-pyrrolidin-1- | 10⁻³ mol | 10⁻³ mol | 10⁻³ mol | 10⁻³ mol | 10⁻³ mol |
| ylethyl)benzene-1,4-diamine | | | | | |
| dihydrochloride | | | | | |
| 5-Amino-2-methylphenol | 10⁻³ mol | | | | |
| 1H-Indol-6-ol | | 10⁻³ mol | | | |
| 2-Aminopyridin-3-ol | | | 10⁻³ mol | | |
| 2-(2,4- | | | | 10⁻³ mol | |
| Diaminophenoxy)ethanol | | | | | |
| hydrochloride | | | | | |
| 3-Amino-2-chloro-6- | | | | | 10⁻³ mol |
| methylphenol hydrochloride | | | | | |
| Dye support (2) | (*) | (*) | (*) | (*) | (*) |
| Demineralized water qs | 100 g | 100 g | 100 g | 100 g | 100 g |

| | |
|---|---|
| (*): dye support (1) pH 7 | |
| 96° Ethyl alcohol | 20.8 g |
| Sodium metabisulfite as an aqueous 35% solution | 0.23 g AM |
| Pentasodium salt of diethylenetriaminepentaacetic acid as | 0.48 g AM |
| an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| Na₂HPO₄ | 0.28 g |
| KH₂PO₄ | 0.46 g |

At the time of use, each composition was mixed with an equal weight of 20-volumes aqueous hydrogen peroxide solution (6% by weight). A final pH of 7 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

Example

| | 65 | 66 | 67 | 68 | 69 |
|---|---|---|---|---|---|
| Shade observed | red- | red | red | strong | violet- |
| | brown | | | blue- | grey |
| | | | | grey | |

### Examples 70 to 72

### Dyeing in Basic Medium

The dye compositions below were prepared:

| | Example | | | | | |
|---|---|---|---|---|---|---|
| | 70 | | 71 | | 72 | |
| 2-Methoxy-6-(2-pyrrolidin-1- | 10⁻³ | mol | 10⁻³ | mol | 10⁻³ | mol |
| ylethyl)benzene-1,4- | | | | | | |
| diamine dihydrochloride | | | | | | |
| 5-Amino-2-methylphenol | 10⁻³ | mol | | | | |
| 2-(2,4-Diaminophenoxy)- | | | 10⁻³ | mol | | |
| ethanol hydrochloride | | | | | | |
| 3-Amino-2-chloro-6- | | | | | 10⁻³ | mol |
| methylphenol hydrochloride | | | | | | |
| Dye support (1) | (*) | | (*) | | (*) | |
| Demineralized water qs | 100 | g | 100 | g | 100 | g |

(*): dye support (2) pH 9.5

| | |
|---|---|
| 96° Ethyl alcohol | 20.8 g |
| Sodium metabisulfite as an aqueous 35% solution | 0.23 g AM |
| Pentasodium salt of diethylenetriaminepentaacetic | 0.48 g AM |
| acid as an aqueous 40% solution | |
| C₈-C₁₀ Alkyl polyglucoside as an aqueous 60% solution | 3.6 g AM |
| Benzyl alcohol | 2.0 g |
| Polyethylene glycol containing 8 ethylene oxide units | 3.0 g |
| NH₄Cl | 4.32 g |
| Aqueous ammonia containing 20% NH₃ | 2.94 g |

At the time of use, each composition was mixed with an equal weight of 20-volumes aqueous hydrogen peroxide solution (6% by weight). A final pH of 9.5 was obtained.

Each mixture obtained was applied to locks of grey hair comprising 90% white hairs. After a leave-on time of 30 minutes, the locks were rinsed, washed with a standard shampoo, rinsed again, and then dried.

The shades obtained are given in the table below:

| | Example | | |
|---|---|---|---|
| | 70 | 71 | 72 |
| Shade observed | red-brown | brown | violet-grey |

What is claimed is:

1. A para-phenylenediamine compound chosen from compounds of formula (I) and physiologically acceptable solvates and acid addition salts thereof: 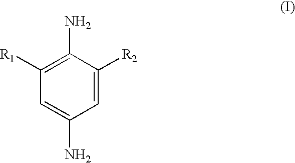
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
with the proviso that the para-phenylenediamine compound is not chosen from:

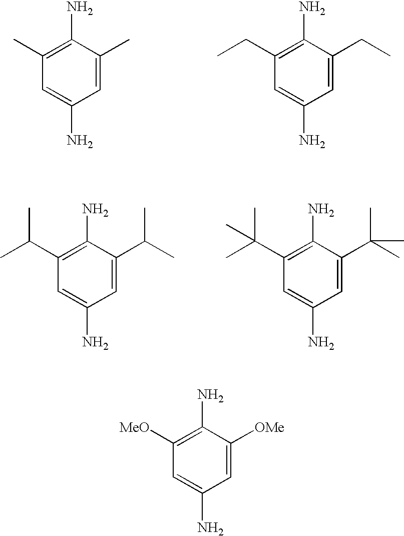

2. The compound of claim 1, wherein R₁ and R₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholinyl, and 1-imidazoyl radicals.

3. The compound of claim 1, wherein R₁ and R₂, which may be identical or different, are chosen from:
methyl, ethyl, n-propyl, isopropyl, sec-butyl, isobutyl, tert-butyl, and (2,2′dimethyl)butyl radicals;
methoxybutyl, aminomethyl, (3-dimethylamino)propyl, imidazolo-n-propyl, and pyrrolidinoethyl radicals;
methoxy, hydroxymethyl, α-hydroxyethyl, β-hydroxyethyl, and β-hydroxyisopropyl radicals;
carboxylic radicals; and
amido radicals.

4. The compound of claim 1, chosen from the following compounds and the physiologically acceptable salts and solvates thereof:

| | |
|---|---|
| 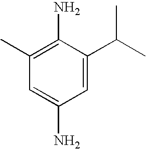 | 2-Isopropyl-6-methylbenzene-1,4- diamine |
| 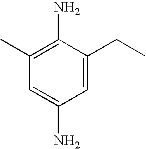 | 2-Ethyl-6-methylbenzene-1,4- diamine |
| 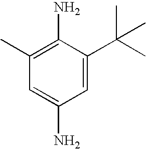 | 2-tert-Butyl-6-methylbenzene-1,4- diamine |
| 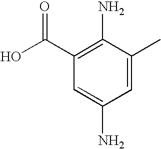 | 2,5-Diamino-3-methylbenzoic acid |
| 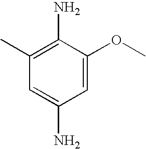 | 2-Methoxy-6-methylbenzene-1,4- diamine |
| 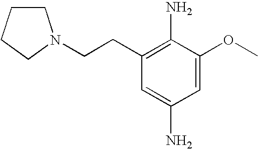 | 2-Methoxy-6-(2-pyrrolidin-1-ylethyl)- benzene-1,4-diamine |
| 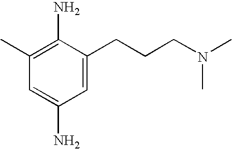 | 2-(3-Dimethylaminopropyl)-6- methyl-benzene-1,4-diamine |
| 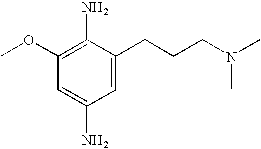 | 2-(3-Dimethylaminopropyl)-6- methoxybenzene-1,4-diamine |
| 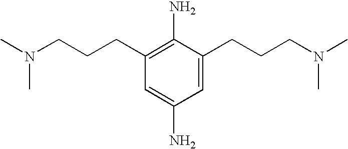 | 2,6-Bis-(3-dimethylaminopropyl)- benzene-1,4-diamine |
| 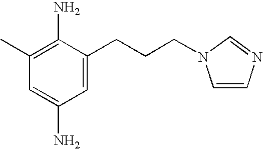 | 2-(3-Imidazol-1-ylpropyl)-6-methyl- benzene-1,4-diamine |
| 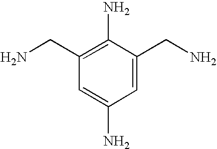 | 2,6-Bisaminomethylbenzene-1,4- diamine |
| 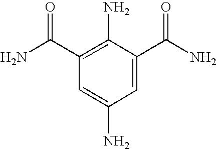 | 2,5-Diaminoisophthalamide |
| 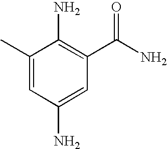 | 2,5-Diamino-3-methylbenzamide |
| 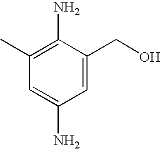 | (2,5-Diamino-3-methylphenyl)methanol |
| 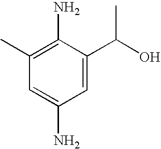 | 1-(2,5-Diamino-3-methylphenyl)ethanol |
| 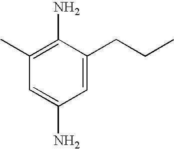 | 2-MethyI-6-propylbenzene-1,4-diamine |
| 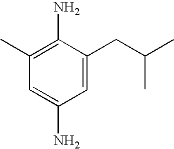 | 2-Isobutyl-6-methylbenzene-1,4- diamine |
| 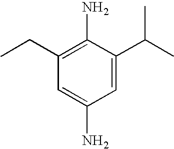 | 2-Ethyl-6-isopropylbenzene-1,4- diamine |
| 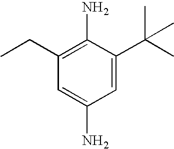 | 2-tert-Butyl-6-ethylbenzene-1,4- diamine |
| 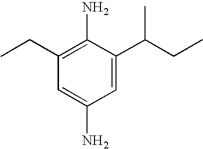 | 2-sec-Butyl-6-ethylbenzene-1,4- diamine |
| 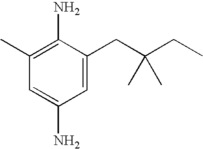 | 2-(2,2-Dimethylbutyl)-6-methyl- benzene-1,4-diamine |
| 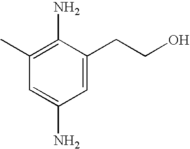 | 2-(2,5-Diamino-3-methylphenyl)ethanol |
| 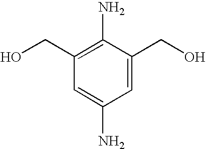 | (2,5-Diamino-3-hydroxymethyl- phenyl)methanol |
| 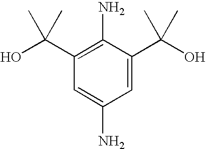 | 2-[2,5-Diamino-3-(1-hydroxy-1- methylethyl)phenyl]propan-2-ol |
| 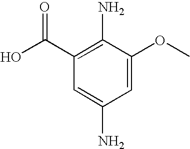 | 2,5-Diamino-3-methoxybenzoic acid |
| 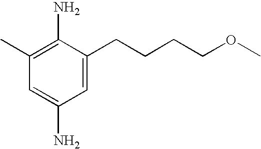 | 2-(4-Methoxybutyl)-6- methylbenzene-1,4-diamine |

5. The compound of claim 1, chosen from 2-isopropyl-6-methylbenzene-1,4-diamine, 2-ethyl-6-methylbenzene-1,4-diamine, 2-tert-butyl-6-methylbenzene-1,4-diamine, 2,5-diamino-3-methylbenzoic acid, 2-methoxy-6-methylbenzene-1,4-diamine, 2-methoxy-6-(2-pyrrolidin-1-ylethyl)benzene-1,4-diamine, 2-(3-dimethylaminopropyl)-6-methylbenzene-1,4-diamine, 2-(3-imidazol-1-ylpropyl)-6-methylbenzene-1,4-diamine, 2,5-diaminoisophthalamide, 2,5-diamino-3-methylbenzamide, 1-(2,5-diamino-3-methylphenyl)-ethanol, 2-methyl-6-propylbenzene-1,4-diamine, 2-isobutyl-6-methylbenzene-1,4-diamine, 2-(2,5-diamino-3-methylphenyl)ethanol, and the physiologically acceptable salts and solvates thereof.

6. A process for synthesizing a para-phenylenediamine compound of formula (I): 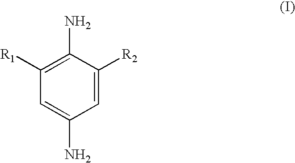
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
the process comprising reducing a corresponding intermediate compound chosen from compounds of formulas (II), (III), and (IV): 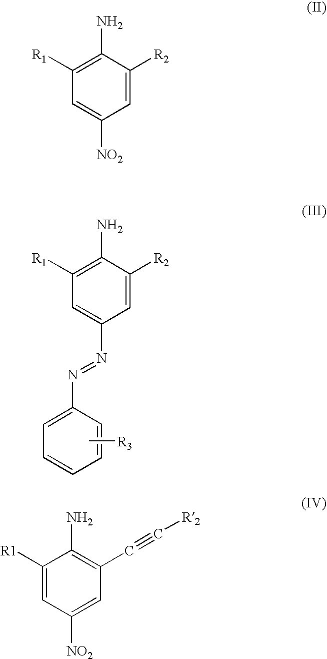
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
R′₂ is chosen from:
linear or branched C₁-C₄ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals; and
R₃ is chosen from hydrogen and sulfonic groups,
with the proviso that the compound of formula (I) is not chosen from:

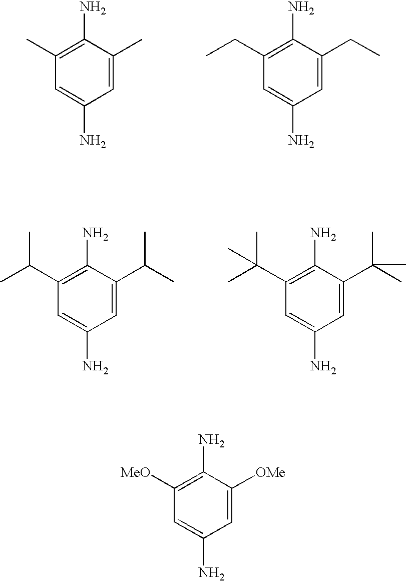

7. The process of claim 6, wherein R₁, R₂, and R′₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholinyl, and 1-imidazoyl radicals.

8. An intermediate compound of formula (II): 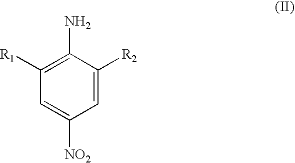
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
with the proviso that the intermediate compound is not chosen from:

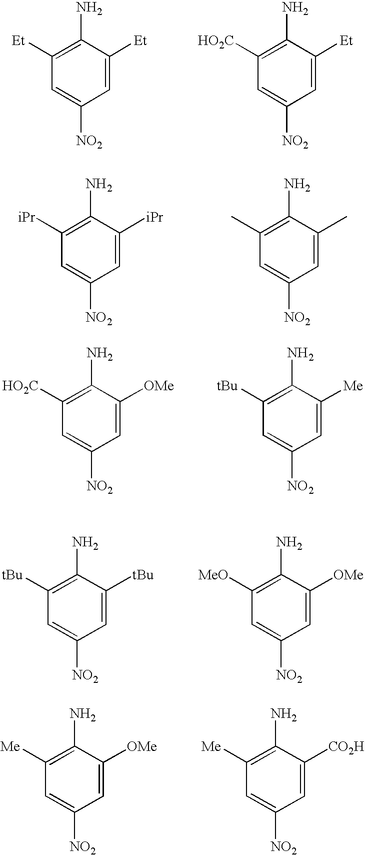

9. The compound of claim 8, wherein the cyclic rings are chosen from 1-pyrrolidinyl, 1-cyclohexylaminyl, 1-piperazinyl, 1-diazepanyl, 1-morpholinyl, and 1-imidazoyl.

10. An intermediate compound of formula (III): 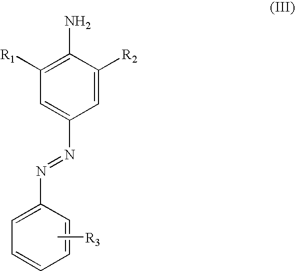
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals; and
R₃ is chosen from hydrogen and sulfonic groups;
with the proviso that the intermediate compound is not chosen from:

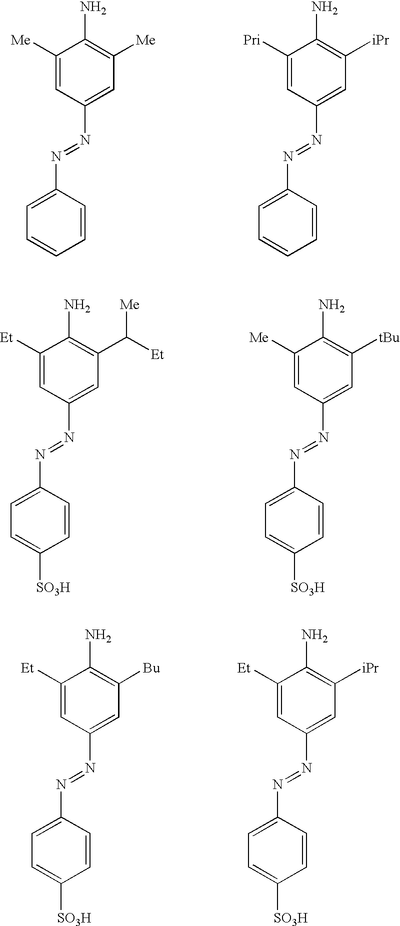

11. The compound of claim 10, wherein R₁ and R₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholinyl, and 1-imidazoyl radicals.

12. An intermediate compound of formula (IV): 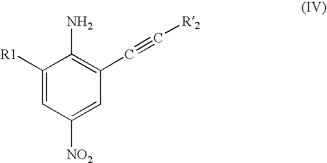
wherein:
R₁ is chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
and R′₂ is chosen from:
linear or branched C₁-C₄ alkyl radical optionally substituted with a C₁-C₆ alkoxy, hydroxyl, amino, (C₁-C₆)alkylamino or (C₁-C₆)dialkylamino radical or alternatively with a cyclic radical such as: 1-pyrrolidinyl, 1-cyclohexylaminyl, 1-piperazinyl, 1-diazepanyl, 1-morpholinyl, 1-imidazoyl, this ring possibly being substituted with one or more alkyl, hydroxyl or amino groups; and
linear or branched C₁-C₆ alkoxy, C₁-C₆ monohydroxyalkoxy, and C₁-C₆ polyhydroxyalkoxy radicals;
with the proviso that the intermediate compound is not chosen from:

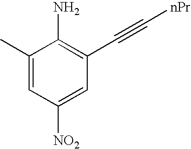

13. The compound of claim 12, wherein R₁ and R′₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholinyl, and 1-imidazoyl radicals.

14. A composition for the oxidation dyeing of keratin fibers, comprising, in a suitable dyeing medium, at least one para-phenylenediamine compound chosen from compounds of formula (I) and the physiologically acceptable solvates and acid addition salts thereof: 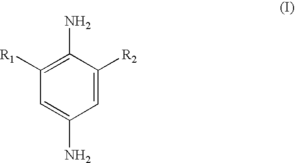
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
with the proviso that the at least one para-phenylenediamine compound is not chosen from:

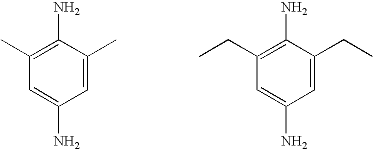

15. The composition of claim 14, wherein R₁ and R₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholinyl, and 1-imidazoyl radicals.

16. The composition of claim 14, wherein R₁ and R₂, which may be identical or different, are chosen from:
methyl, ethyl, n-propyl, isopropyl, sec-butyl, isobutyl, tert-butyl, and (2,2′dimethyl)butyl radicals;
methoxybutyl, aminomethyl, (3-dimethylamino)propyl, imidazolo-n-propyl, and pyrrolidinoethyl radicals;
methoxy, hydroxymethyl, α-hydroxyethyl, β-hydroxyethyl, and β-hydroxyisopropyl radicals;
carboxylic radicals; and
amido radicals.

17. The composition of claim 14, wherein the at least one para-phenylenediamine compound is chosen from: 2-isopropyl-6-methylbenzene-1,4-diamine, 2-ethyl-6-methylbenzene-1,4-diamine, 2,6-diisopropylbenzene-1,4-diamine, 2-tert-butyl-6-methylbenzene-1,4-diamine, 2,5-diamino-3-methylbenzoic acid, 2-methoxy-6-methyl-benzene-1,4-diamine, 2-methoxy-6-(2-pyrrolidin-1-ylethyl)benzene-1,4-diamine, 2-(3-dimethylaminopropyl)-6-methylbenzene-1,4-diamine, 2-(3-dimethylaminopropyl)-6-methoxybenzene-1,4-diamine, 2,6-bis(3-dimethylaminopropyl)benzene-1,4-diamine, 2-(3-imidazol-1-ylpropyl)-6-methylbenzene-1,4-diamine, 2,6-bis(aminomethyl)benzene-1,4-diamine, 2,5-diaminoisophthalamide, (2,5-diamino-3-methylphenyl)methanol, 2,5-diamino-3-methylbenzamide, 1-(2,5-diamino-3-methylphenyl)ethanol, 2-methyl-6-propylbenzene-1,4-diamine, 2-isobutyl-6-methylbenzene-1,4-diamine, 2-ethyl-6-isopropylbenzene-1,4-diamine, 2-tert-butyl-6-ethylbenzene-1,4-diamine, 2-sec-butyl-6-ethylbenzene-1,4-diamine, 2-(2,2-dimethylbutyl)-6-methylbenzene-1,4-diamine, 2-(2,5-diamino-3-methylphenyl)ethanol, (2,5-diamino-3-hydroxymethylphenyl)methanol, 2-[2,5-diamino-3-(1-hydroxy-1-methylethyl)phenyl]propan-2-ol, 2,5-diamino-3-methoxybenzoic acid, 2-(4-methoxybutyl)-6-methylbenzene-1,4-diamine, and the physiologically acceptable salts and solvates thereof.

18. The composition of claim 14, wherein the at least one para-phenylenediamine compound of formula (I) is chosen from 2-isopropyl-6-methylbenzene-1,4-diamine, 2-ethyl-6-methylbenzene-1,4-diamine, 2,6-diisopropylbenzene-1,4-diamine, 2-tert-butyl-6-methylbenzene-1,4-diamine, 2,5-diamino-3-methylbenzoic acid, 2-methoxy-6-methylbenzene-1,4-diamine, 2-methoxy-6-(2-pyrrolidin-1-ylethyl)benzene-1,4-diamine, 2-(3-dimethylaminopropyl)-6-methyl-benzene-1,4-diamine, 2-(3-imidazol-1-ylpropyl)-6-methylbenzene-1,4-diamine, 2,5-diaminoisophthalamide, 2,5-diamino-3-methylbenzamide, 1-(2,5-diamino-3-methylphenyl)ethanol, 2-methyl-6-propylbenzene-1,4-diamine, 2-isobutyl-6-methylbenzene-1,4-diamine, 2-(2,5-diamino-3-methylphenyl)ethanol, and the physiologically acceptable salts and solvates thereof.

19. The composition of claim 14, wherein the at least one para-phenylenediamine compound is present in the composition in an amount ranging from 0.001% to 10% by weight relative to the total weight of the composition.

20. The composition of claim 19, wherein the at least one para-phenylenediamine compound is present in the composition in an amount ranging from 0.05% to 6% by weight relative to the total weight of the composition.

21. The composition of claim 20, wherein the at least one para-phenylenediamine compound is present in the composition in an amount ranging from 0.1% to 3% by weight relative to the total weight of the composition.

22. The composition of claim 14, wherein the suitable dyeing medium is chosen from water and mixtures of water and at least one organic solvent chosen from C₁-C₄ lower alkanols, polyols, polyol ethers, aromatic alcohols, and mixtures thereof.

23. The composition of claim 14, having a pH ranging from 3 to 13.

24. The composition of claim 14, further comprising at least one additional oxidation base chosen from para-phenylenediamines other than the para-phenylenediamines of formula (I), bis(phenyl)alkylenediamines, para-aminophenols, ortho-aminophenols, heterocyclic bases, and the acid addition salts thereof.

25. The composition of claim 24, wherein the at least one additional oxidation base is present in the composition in an amount ranging from 0.0005% to 12% by weight relative to the total weight of the composition.

26. The composition of claim 14, further comprising at least one coupler and/or at least one direct dye.

27. The composition of claim 26, wherein the at least one couplers is chosen from meta-phenylenediamines, meta-aminophenols, meta-diphenols, monohydroxylated or polyhydroxylated naphthalene derivatives, heterocyclic couplers, and the acid addition salts thereof.

28. The composition of claim 26, wherein the at least one coupler is present in the composition in an amount ranging from 0.0001% to 10% by weight relative to the total weight of the composition.

29. The composition of claim 14, wherein the acid addition salts are chosen from the salts of hydrochloric acid, hydrobromic acid, sulfuric acid, acetic acid, lactic acid, tartaric acid, citric acid, methanesulfonic acid, para-toluenesulfonic acid, benzenesulfonic acid, phosphoric acid, and succinic acid.

30. A process for dyeing keratin fibers comprising
applying at least one dye composition to the fibers for a time that is sufficient to develop a desired coloration, either in air or using at least one oxidizing agent, and optionally in the presence of at least one oxidation catalyst, wherein the at least one dye composition comprises, in a suitable dyeing medium, at least one para-phenylenediamine compound chosen from compounds of formula (I) and the physiologically acceptable solvates and acid addition salts thereof: 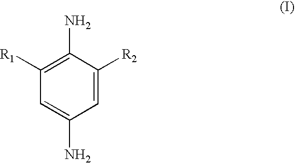
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
with the proviso that the at least one para-phenylenediamine compound is not chosen from:

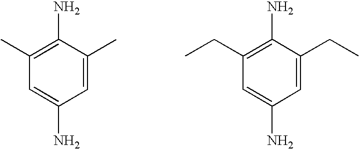

31. The process of claim 30, wherein R₁ and R₂ are independently chosen from linear or branched C₁-C₆ alkyl radicals optionally substituted with at least one cyclic radical comprising a ring, said at least one cyclic radical being chosen from 1-pyrrolidinyl, 1-cyclohexylaminyl, 1-piperazinyl, 1-diazepanyl, 1-morpholyinyl, and 1-imidazoyl radicals.

32. The process of claim 30, wherein the coloration is revealed upon contact with atmospheric oxygen.

33. The process of claim 30, wherein the coloration is revealed at a pH chosen from acidic, neutral, and alkaline pHs, using at least one oxidizing agent that is added just at the time of application to the dye composition or that is present in an oxidizing composition applied simultaneously or sequentially in a separate manner.

34. The process of claim 33, wherein the at least one oxidizing agent is chosen from hydrogen peroxide, urea peroxide, alkali metal bromates, and persalts.

35. The process of claim 34, wherein the persalts are chosen from perborates and persulfates.

36. A multi-compartment device comprising at least one first compartment comprising at least one dye composition and at least one second compartment comprising at least one oxidizing composition, wherein the at least one dye composition comprises, in a suitable dyeing medium, at least one para-phenylenediamine compound chosen from compounds of formula (I) and the physiologically acceptable solvates and acid addition salts thereof: 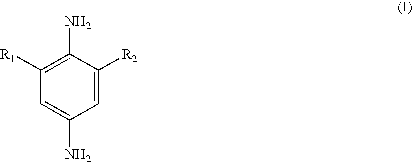
wherein:
R₁ and R₂, which may be identical or different, are chosen from:
linear or branched C₁-C₆ alkyl radicals optionally substituted with at least one radical chosen from C₁-C₆ alkoxy radicals, hydroxyl radicals, amino radicals, (C₁-C₆)alkylamino radicals, (C₁-C₆)dialkylamino radicals, and cyclic radicals comprising a ring that is optionally substituted with at least one group chosen from alkyl, hydroxyl, and amino groups;
linear or branched C₁-C₆ alkoxy, C₁-C₆ monohydroxyalkoxy, and C₁-C₆ polyhydroxyalkoxy radicals;
carboxylic radicals; and
amide radicals;
with the proviso that the at least one para-phenylenediamine compound is not chosen from: [chemical structure]
